(19) United States
(12) Patent Application Publication
(10) Pub. No.: US 2009/0165783 A1
(43) Pub. Date: Jul. 2, 2009

(54) Nebulizer Apparatus and Method

(76) Inventor: Molly McDonald, Auburn, AL (US)

Correspondence Address: LANIER FORD SHAVER & PAYNE P.C., P O BOX 2087, HUNTSVILLE, AL, 35804-2087, US

(21) Appl. No.: 11/936,188

(22) Filed: Dec. 31, 2007

(51) Int. Cl. A61M 15/00

(52) U.S. Cl. 128/200.21

(57) ABSTRACT

A device has a mouthpiece with an inhalation opening and a substantially spheroid container unitarily formed with the mouthpiece, the spheroid container for housing liquid medicine. The device further comprises an air delivery opening coupled to the spheroid container, wherein when air is delivered to the container, the air mixes with the medicine creating vaporized medicine for delivery to a user via the inhalation opening.

### BACKGROUND

A “nebulizer” refers to device that is commonly used to administer concentrated medicine, sometimes referred to as “viscous,” to a patient. The nebulizer delivers a liquid mist form of the viscous to the airways of the patient. Nebulizers are often used to treat, for example, cystic fibrosis, asthma, and other respiratory diseases.

The nebulizer pumps air or oxygen through the concentrated liquid and turns the liquid into a medicated vapor. The medicated vapor is then inhaled by the patient.

### SUMMARY

Embodiments of the present disclosure generally relate to nebulizer apparatuses and methods. In particular, the present disclosure relates to apparatuses; that are used by a patient to take vaporized medicine.

An apparatus in accordance with an embodiment of the present disclosure comprises a mouthpiece comprising an inhalation opening and a substantially spheroid container formed as a unitary piece with the mouthpiece, the spheroid container for housing liquid medicine. The device further comprises an air delivery opening coupled to the spheroid container, wherein when air is delivered to the container, the air mixes with the medicine creating vaporized medicine for delivery to a user via the inhalation opening.

A method in accordance with an embodiment of the present disclosure comprises the steps of pouring liquid medicine in an opening in a substantially spheroid container that is unitarily formed with a hinged mouthpiece and closing the hinged mouthpiece so that the opening is covered by the mouthpiece. The method further comprises the steps of receiving air in the substantially spheroid container and inhaling medicated mist from the mouthpiece created by the combination of the liquid medicine and the received air.

### BRIEF DESCRIPTION OF THE DRAWINGS

The disclosure can be better understood with reference to the following drawings. The elements of the drawings are not necessarily to scale relative to each other, emphasis instead being placed upon clearly illustrating the principles of the disclosure. Furthermore, like reference numerals designate corresponding parts throughout the several views.

FIG. 1 is a diagram illustrating a perspective view of a nebulizer in accordance with an embodiment of the present disclosure.

FIG. 2 is a diagram illustrating a bottom plan view of the nebulizer depicted in FIG. 1.

FIG. 3 is diagram illustrating a cross-sectional view of the nebulizer depicted in FIG. 1.

FIG. 4 is diagram illustrating a side plan view of the nebulizer depicted in FIG. 1 with the nebulizer in an open position.

FIG. 5 is a flowchart illustrated an exemplary method of the nebulizer depicted in FIG. 1 in accordance with an embodiment of the present disclosure.

### DETAILED DESCRIPTION

The present disclosure generally pertains to nebulizer apparatuses and methods. In particular, a nebulizer in accordance with one exemplary embodiment of the present disclosure comprises a spheroid container hingedly coupled to a mouthpiece.

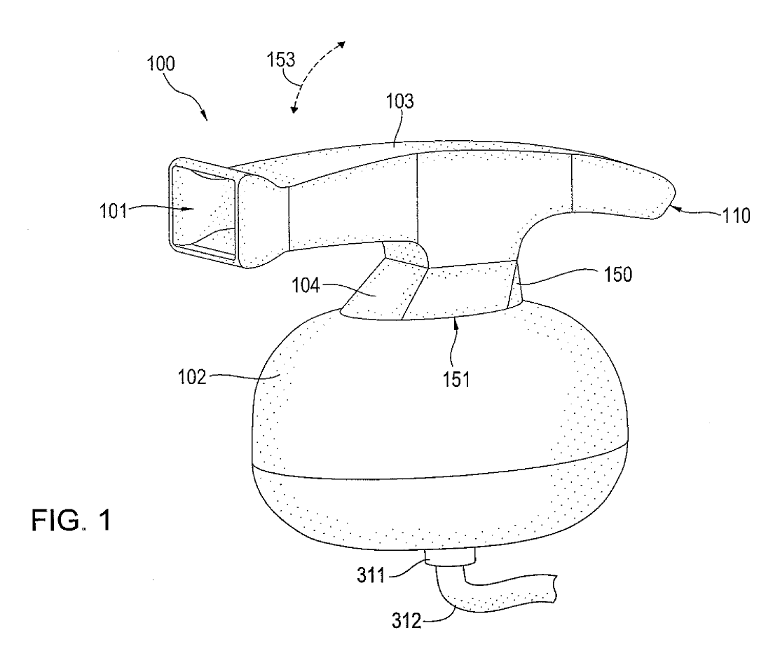

FIG. 1 depicts a perspective view of a nebulizer 100 in accordance with an embodiment of the present disclosure. The nebulizer 100 comprises a spheroid container 102, a neck 104, and a tube-like mouthpiece 103. The tube-like mouth piece 103 comprises a vaporized medicine exit opening 101 and a residual exit opening 110.

In one embodiment, the spheroid container 102, the neck 104, and the tube-like mouthpiece 103 make up a unitary piece. In such an embodiment, the nebulizer 100, including the spheroid container 102, the neck 104, and the tube-like mouthpiece 103 are comprised of a plastic material created as a unitary piece via a plastic molding process. Note that other materials may be used to make the nebulizer 100 in other embodiments.

In such an embodiment, the spheroid container 102 is hingedly separable via a plastic hinge 150 along an intersection 151 of the neck 104 and the container 102. The plastic hinge 150 is unitarily formed with the spheroid container 102 and the neck 104 during the molding process. Note that other types of hinges may be used in other embodiments of the nebulizer 100.

Notably, when a user (not shown) desires to use the nebulizer 100, the user grabs the mouthpiece 103 and rotates the mouthpiece 103 in a direction indicated by reference line 153. Upon such actuation by the user, the neck 104 separates from the container 102 along intersection 151 and the plastic hinge 150 deforms so that the mouthpiece 103 and the neck 104 are separated from the container 102.

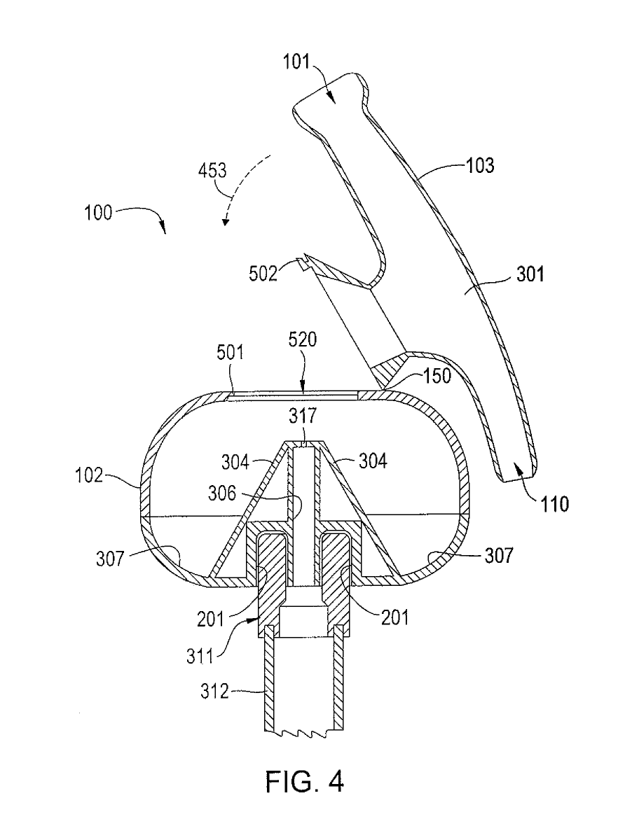

In one embodiment, the mouthpiece 103 and the neck 104 are connected to the spheroid container 102 via the plastic hinge 150 that is a piece of plastic perforated so that the plastic bends at a point (not identified) to allow the neck 104 and the mouthpiece 103 to open in a direction indicated by double reference arrow 153 along the intersection 151. The nebulizer 100 illustrated in an open position is described in more detail with reference to FIG. 4.

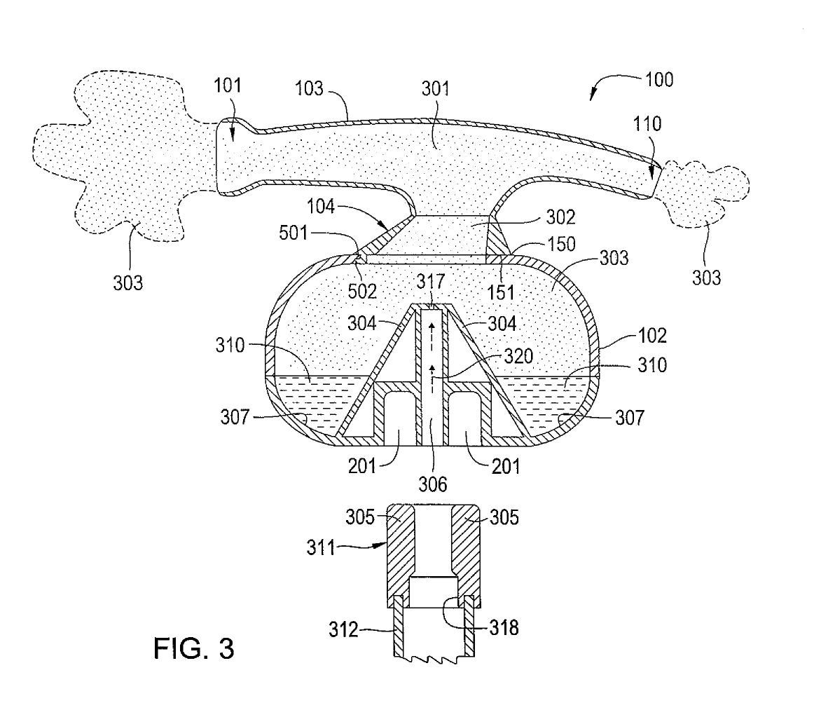

In order for the user to use the nebulizer 100, the user fills the spheroid 102 with liquid medicine (not shown). In this regard, the spheroid container 102 is filled with liquid medicine, described further with reference to FIG. 3, by opening the nebulizer 100 and pouring medicine into the spheroid container 102. As described hereinabove, the nebulizer 100 is opened by the user grabbing the mouthpiece 103 and rotating the neck 104 and the mouthpiece 103 in the direction indicated by reference arrow 153. Once the user pours the medicine into the container 102, the user closes the nebulizer 100 by rotating the mouthpiece 103 and the neck 104 in the opposing direction also indicated by double reference arrow 153, which illustrates the direction of movement of the mouthpiece 103 and the neck 104. In one embodiment, the neck 104 is attached to the container 102 via a locking mechanism, which is described further with reference to FIG. 3.

In addition, the nebulizer 100 is coupled to an air tube 312 via a tube connector 311. Note that the tube 312 is connected at its opposing end to an air-producing device (not shown), such as, for example, and air compressor. The compressor creates an air stream that fills the container 102 to create a mist via evaporation. It produces an aerosol-like mist by breaking down the aqueous molecules/particles of liquid medicine placed in the container, as described further herein.

Once medicine is placed within the container 102 and the neck 104 and the mouthpiece 103 are coupled to the container 102, the nebulizer 100 is ready for use.

During operation, the tube 312 delivers air or oxygen (not shown) to the container 102 via the connector 311. The air or oxygen combines with the liquid medication in the spheroid container 102 to produce a medicated vapor, which is described further with reference to FIG. 3.

Note that the air tube 312 is connected to a device (not shown) that produces a flow of air/oxygen. For example, the air tube 312 may be connected to an air or oxygen pump (not shown) that produces air or oxygen.

A user (not shown) of the nebulizer 100 places his mouth on the vaporized medicine exit opening 101 and inhales. When the user inhales, the medicated vapor is delivered to the user's lungs for treatment of, for example, cystic fibrosis, asthma, or other respiratory disease. Any excess medicated vapor flows out of the mouthpiece 103 through the residual exit opening 110.

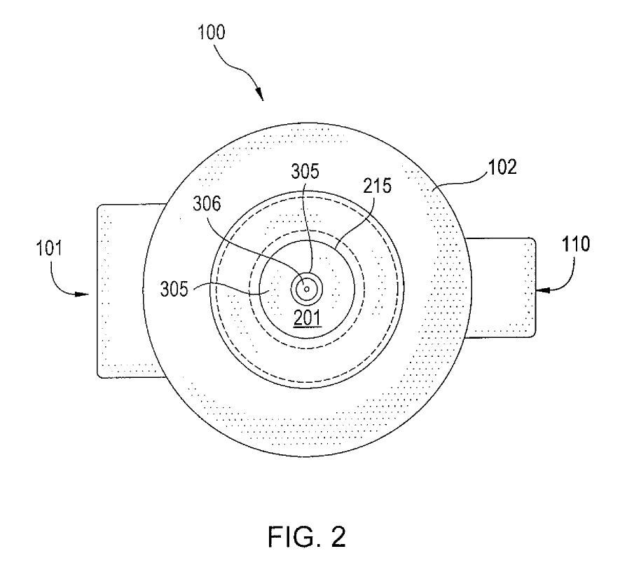

The nebulizer 100 is connected via a connector 311 to a tube 312. Connection of the tube 312 via the connector 311 is described in more detail with reference to FIGS. 2 and 3. Further, the tube 312 is connected to an air-producing or oxygen-producing device (not shown). Note that other connectors may be used in other embodiments to connect the nebulizer 100 to the air tube 312. In addition, other types of air delivery devices, other than the air tube 312 may be used to deliver air to the spheroid container 102 of the nebulizer 100.

Note that the spheroid container 102 depicted in FIG. 1 has a varying radius and in most respects is not consistently symmetrical from every vantage point. In this regard, the spheroid container 102 has an oval cross-section. However, in other embodiments, the radius of the spheroid container may be constant such that the cross-section of the spheroid container 102 is a circle.

FIG. 2 depicts an underside plan view of the nebulizer 100 depicted in FIG. 1. As described hereinabove with reference to FIG. 1, the nebulizer 100 connects to the plastic tube 312 (FIG. 1) via the connector 311 (FIG. 1). FIG. 2 illustrates an embodiment of the spheroid container 102 so that such connection is made.

In this regard, the underside of the nebulizer 100 comprises a circular channel 201. The circular channel 201 is formed by concentric circular walls 215 and 305. In addition, the nebulizer 100 comprises an air/oxygen delivery channel 306 opening on the underside of the spheroid container 102. The delivery channel 306 provides an interface for receiving air (not shown), which is delivered to the inside of the spheroid container 102.

The delivery channel receives the tube 312 (FIG. 1) via the connector 311 (FIG. 1), which is described further with reference to FIG. 3. In this regard, the tube 312 couples to the connector 311, and the connector 311 fits within the circular channel 201, thereby securely interfacing the tube 312 to the air delivery channel 306. Air delivered to the channel 306 travels through the channel 306 into the spheroid container 102.

The delivered air mixes with the medicine (not shown) in the spheroid container 102, thereby generating a medicated vapor that can be inhaled by the user. The user places his mouth over the opening 101 and inhales. Any residual medicated vapor that is not inhaled by the user exits out the residual exit opening 110.

FIG. 3 depicts a cross-sectional view of the nebulizer 100 when the nebulizer 100 is in a “closed position,” i.e., after medicine has been poured into the container 102 and the nebulizer 100 is ready for use. Hereinafter, the “closed position” refers to that position of the neck 104 and the mouthpiece 103 when the nebulizer 100 is ready for use. Note that FIG. 4 depicts the nebulizer 100 wherein the nebulizer 100 is in an “open position,” which is described further herein. Further, FIG. 3 depicts a cross-sectional view of the connector 311.

The tube 312 connects to the nebulizer 100 via the connector 311. In this regard, the connector 311 comprises an opening 318 in which the tube 312 is inserted. The opening 318 is formed by a circular wall 305. The circular wall 305 fits within the circular channel 201 thereby interfacing the tube 312 with the air delivery channel 306.

The nebulizer 100 further comprises an internal cone structure 304 that houses the circular channel 201 and the air delivery channel 306. Such cone structure 304 is internal to the spheroid container 102. In addition, the neck 104 forms a channel 302, and the mouthpiece 103 forms a channel 301 that interfaces to the medicated vapor exit opening 101 and the residual exit opening 110.

The air/oxygen deliver channel 306 extends through the length of the cone 304 and an opening 317 in the cone 304 allows air/oxygen 321 into the container 102 of the nebulizer 100. During operation, medicine 310 resides at the bottom 307 of the container 102. When the air/oxygen 321 enters the container 102 through the opening 317, the air/oxygen 321 mixes with the liquid medicine 310 to form a medicated vapor 303.

When the user (not shown) places his mouth (not shown) over the medicated vapor exit opening 101 of the mouthpiece 103 and inhales, the medicated vapor 303 flows from the spheroid container 102, through the neck channel 302 and into the mouthpiece channel 301. The medicated vapor 303 enters the user's lungs of the user when the user inhales through the exit opening 101 to treat, for example, cystic fibrosis, asthma, or other type of respiratory disease. Note that the liquid medicine 310 may be, for example, albuterol sulfate.

The nebulizer 100 further comprises a c-shaped locking mechanism 502 in the neck 104. The container 102 further comprises a flange 501. When the nebulizer 100 is in the closed position, the c-shaped locking mechanism 502 snaps onto the flange 501 to retain the nebulizer 100 in the closed position.

FIG. 4 shows the nebulizer 100 in the open position, as described hereinabove. The neck 104 of the nebulizer 100 is hingedly coupled to the container 102 via a perforated plastic hinge 150. Note that the hinge 150 is not a separate component but is a piece of the unitary plastic of the nebulizer 100 that is perforated such that the neck is hingedly coupled to the container 102.

Furthermore, as described with reference to FIG. 3, the container 102 comprises a flange 501 that protrudes slightly. This flange 501 is part of the plastic molded container 102. Further, the neck 104 comprises the c-shaped locking mechanism 502. To place the nebulizer 100 in the closed position, as described hereinabove, the user applies force to the mouthpiece 103 such that the mouthpiece 103 moves in the direction indicated by reference number 453. The c-shaped locking mechanism 502 couples to the flange 501 in order to retain the nebulizer 100 in the closed position.

When the nebulizer 100 is in the open position as shown in FIG. 4, medicine 310 (FIG. 3) is introduced into the container 102 through an opening 520 in the spheroid container 102. The medicine 310 flows down the outside of the cone 304 such that the cone 304 directs the medicine 310 to the bottom 307 of the container 102. The medicine 310 then resides at the bottom 307 of the container 102 until it is vaporized by the air/oxygen 321 (FIG. 3) provided to the container 102 via the air tube 312 via the connector 311.

Note that in one embodiment, the nebulizer 100 comprises a seal (not shown). The seal is fixed at the intersection 151 between the neck 104 and the container 102. Such seal would decrease the amount of medicine vapor 303 from escaping where the neck 104 and the container 102 meet. Such a seal may be comprised of an elastomeric material, such as, for example, silicon, viton®, rubber, and the like.

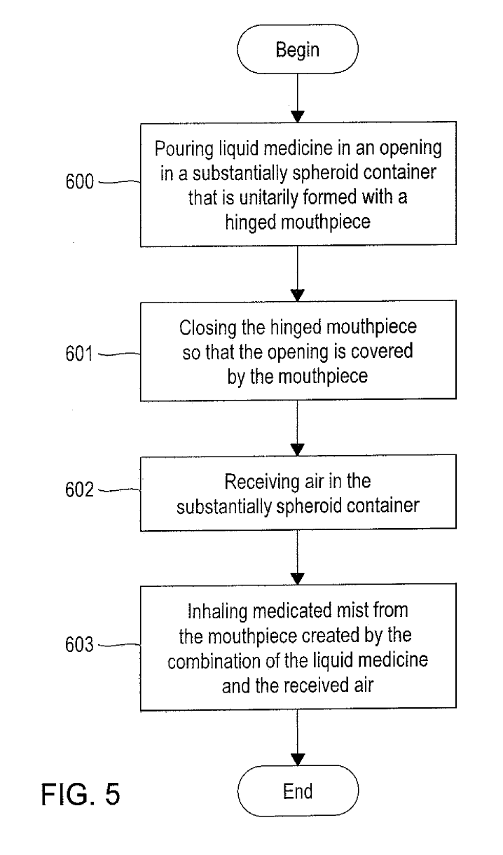

A method in accordance with an embodiment of the present disclosure is illustrated in FIG. 5.

The method comprises the first step of pouring the liquid medicine 310 (FIG. 3) into the substantially spheroid container 102 (FIG. 3) via the opening 520 (FIG. 4), as indicated in step 600. Note that once the liquid medicine 310 is introduced to the container 102 through the opening 520, the cone structure 304 passively directs the medicine to the bottom 307 of the container 102.

Once the liquid medicine 310 is in the container 102, the next step is closing the hinged mouthpiece 103 (FIG. 3) so that the opening 520 is covered by the mouthpiece 103, as indicated in step 601. Notably, the mouthpiece 103 and the container 102 are a unitary piece connected via a plastic hinge 150 that is molded to the neck 104 and the container 102.

Once the mouthpiece 103 is closed and locked via the c-shaped locking mechanism 502 and the flange 501, the container then receives air/oxygen 320 (FIG. 3) in the substantially spheroid container. As described hereinabove, the air/oxygen 320 may be produced by an air/oxygen pump (not shown).

Once the air/oxygen 320 is introduced to the container 102, the user may inhale medicated vapor 303 (FIG. 3) from the mouthpiece 103 created by the combination of the liquid medicine 310 and the received air/oxygen 320, as indicated in step 603. The air/oxygen 320 combines with the liquid medicine 310 to form the medicated vapor 303. The medicated vapor 303 travels through the channel 302 and the channel 301 out the exit opening 101. Any residual medicated vapor 303 exits the nebulizer 100 through the exit opening 110.

1. A device, comprising:
a mouthpiece comprising an inhalation opening;
a substantially spheroid container formed with the mouthpiece, the spheroid container for housing liquid medicine; and
an air delivery opening coupled to the spheroid container, wherein when air is delivered to the container, the air mixes with the medicine creating vaporized medicine for delivery to a user via the inhalation opening.

2. The device of claim 1, wherein the spheroid container has a constant radius.

3. The device of claim 1, wherein the spheroid container has a varying radius.

4. The device of claim 1, wherein the mouthpiece comprises a residual exit opening for removal of residual vaporized medicine.

5. The device of claim 1, wherein the mouthpiece is hingedly coupled to the container.

6. The device of claim 5, wherein the mouthpiece is hingedly coupled to the container via a unitary perforated plastic hinge.

7. The device of claim 1, further comprising a c-shaped locking mechanism formed in the mouthpiece.

8. The device of claim 7, further comprising a locking flange formed in the container.

9. The device of claim 8, wherein the c-shaped locking mechanism and the flange couple to enclose the medicine in the container.

10. The device of claim 1, further comprising a cone formed in the container, the cone comprising a spheroid aperture formed by a first and second wall for receiving an air delivery connector, the air delivery connector coupled to a tube.

11. The device of claim 1, further comprising an air delivery channel for coupling to the tube such that air flows through the channel to the container.

12. The device of claim 1, further comprising a seal between the container and the mouthpiece

13. A method, comprising:
pouring liquid medicine in an opening in a substantially spheroid container that is unitarily formed with a hinged mouthpiece;
closing the hinged mouthpiece so that the opening is covered by the mouthpiece;
receiving air in the substantially spheroid container; and
inhaling medicated mist from the mouthpiece created by the combination of the liquid medicine and the received air.

14. The method of claim 13, further comprising connecting the spheroid container to an air tube.

15. The method of claim 14, wherein the spheroid container comprises a circular channel and the tube is attached to a connector having a circular protrusion further comprising the step of connecting the tube to the spheroid container by inserting the protrusion into the channel.

16. The method of claim 13, wherein the spheroid container comprises a flange and the mouthpiece comprises a c-shaped locking mechanism.

17. The method of claim 16, further comprising the step of fixedly coupling the mouthpiece to the container by inserting the flange in the c-shaped locking mechanism.

18. The method of claim 13, further comprising the step of distributing the liquid medicine throughout the spheroid container via a fixed cone structure internal to the spheroid container.

19. The method of claim 13, further comprising sealing the intersection of the mouthpiece and the container.

20. A nebulizer device, comprising:
a mouthpiece comprising an inhalation opening;
a substantially spheroid container formed with the mouthpiece, the spheroid container for housing liquid medicine; and
means for delivering air coupled to the spheroid container, wherein when air is delivered to the container, the air mixes with the medicine creating vaporized medicine for delivery to a user via the inhalation opening.
